(19) United States
(12) Patent Application Publication
(10) Pub. No.: US 2006/0237554 A1
(43) Pub. Date: Oct. 26, 2006

(54) Dispenser for animal repellant effluent

(76) Inventor: Charles Colarusso, Brockton, MA (US)

Correspondence Address: JOHN BRANDT, 60 THAXTER ST., HINGHAM, MA, 02043, US

(21) Appl. No.: 11/174,390

(22) Filed: Jul. 5, 2005

Related U.S. Application Data
(60) Provisional application No. 60/674,062, filed on Apr. 22, 2005.

(51) Int. Cl. A24F 25/00; A61L 9/04

(52) U.S. Cl. 239/34; 239/46; 239/51.5

(57) ABSTRACT

A dispenser for animal repellent effluent is disclosed consisting of a chemical repellent containing compartment having a plurality of vents, a compartment cover to protect the repellent from the weather and a positioning or attaching device to fix the dispenser at a selected location. The vents provide for the dissemination of repellent effluent about the location such as a trashcan, dumpster, or exterior of a building.

### CROSS REFERENCE TO RELATED APPLICATIONS

This application is based on Provisional Application Ser. No. 60/674,062 filed Apr. 22, 2005.

### BACKGROUND OF THE INVENTION

1. Field of the Invention

The invention resides in the field of devices for warding off unwanted animals by the release to the environment of an effluent offensive to the animals.

2. Description of the Prior Art

Several devices for emitting a chemical effluent repellent to animals exist in the prior art. The purpose of these is to provide a container for an animal repellent chemical, mothballs for example, which may be situated in an area where animal pests are likely to gather to discourage their presence by an odor offensive to them. Examples are U. S. Pat. No. 3,840,145, Almanza; U.S. Pat. No. 4,804,142, Riley; U.S. Pat. No. 5,379,545, Gall et al.; and U.S. Pat. No. 5,988,520, Bitner.

The invention disclosed herein provides unique and novel features over the prior art known to applicant, in particular, the location and secure placement of the dispensers which enhance their use.

### SUMMARY OF THE INVENTION

The invention may be summarized as an animal repellent effluent emitter or dispenser which is attachable or securable at a selected location to which animals are likely to be attracted. The apparatus includes a chamber for holding the repellent substance, moth balls for example, a chamber cover for protecting the repellent from the weather, and means for positioning, attaching or securing the dispenser. Vents in the chamber are provided to introduce the repellent effluent into the atmosphere.

A plurality of embodiments are disclosed herein. The dispenser may be a separate attachable unit or may be molded directly into the structure of a trashcan or dumpster. It may also take the form of a pail with an interior shelf coupled with a stake for holding the dispenser in place by sinking the stake into the ground.

These and other features and advantages, as well as the detailed structure of the invention will become more evident from the description of the preferred embodiment taken in conjunction with the drawings which follows.

### DESCRIPTION OF THE DRAWINGS

FIG. 1 is a cross-sectional view of a first preferred embodiment of the invention;

FIG. 2 is a cross-sectional view of an alternative mounting position of the dispenser of FIG. 1;

FIG. 3 is a perspective view of an additional placement of the dispenser of FIG. 1;

FIG. 4 is a cross-sectional view of an additional mounting arrangement of the dispenser of FIG. 1;

FIG. 5 is a cross-sectional view of an additional embodiment of the invention;

FIG. 6 is a partially cross-sectional front view of another embodiment of the invention;

FIG. 7 is a partially cross-sectional front view of a further embodiment of the invention;

FIG. 8 is a plan view of the embodiment of FIG. 7; and

FIG. 9 is a front view of another embodiment of the invention.

### DESCRIPTION OF THE PREFERRED EMBODIMENT

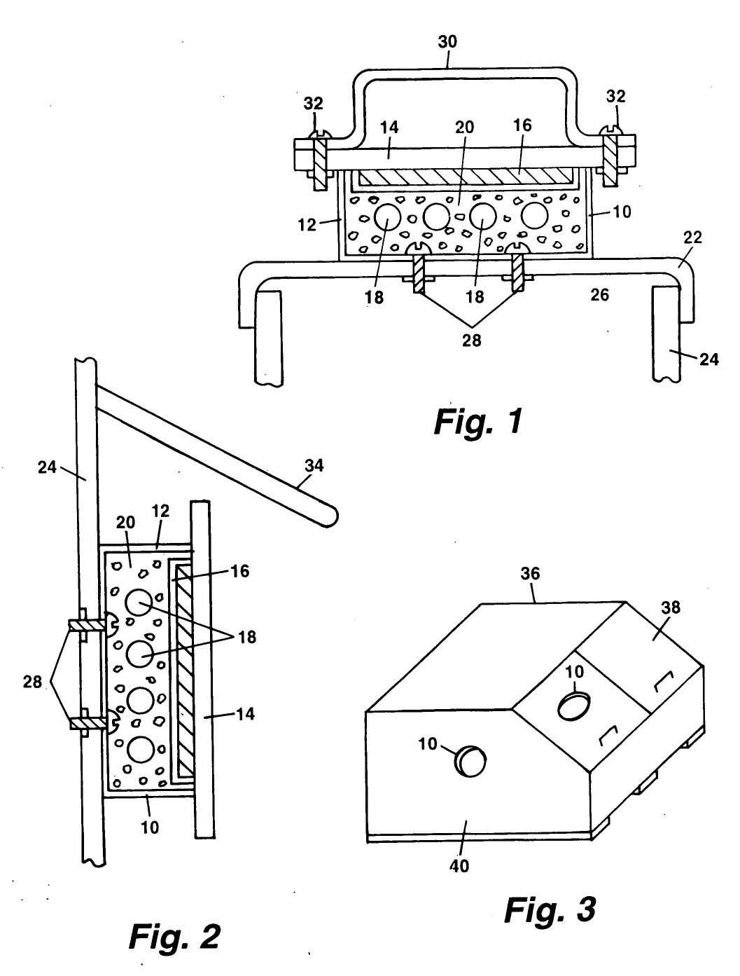

Referring first to FIG. 1, there is shown a cross-sectional view of a first preferred embodiment of the invention in which dispenser 10 of any appropriate shape is composed of a lower compartment 12 for containing a chemical animal repellent and an upper cover 14 for securing and protecting the repellent. The cover may be held in place by the force fit of a flange 16 as shown or by, for example, matching threads, clips, Velcro strips, or similar such mechanical means. Ports 18 are placed about the circumference of compartment 12 to release the vapors from chemical 20, a substance such as moth crystals, disposed in the compartment.

Dispenser 10 is attached to cover 22 of trashcan 24 by, for example, a strong double-sided adhesive tape 26 or other mechanical means such as nut and bolt sets 28. Additionally, a handle 30 may be attached to cover 14 by, for example, nut and bolt sets 32 to lift both dispenser cover 14 and/or trash can cover 22 depending upon how cover 14 is attached to container 12.

Referring next to FIG. 2, the dispenser of FIG. 1 is shown combined with the body of can 24 and in particular is mounted under trash can handle 34 so that, in addition to the protection provided by cover 14, the entire dispenser is shielded from the weather by handle 34 when it is secured in this position.

Referring next to FIG. 3, dispenser 10 is illustrated mounted on dumpster36 on both door 38 and side 40. The same means as described above may be used to secure the dispenser to the dumpster.

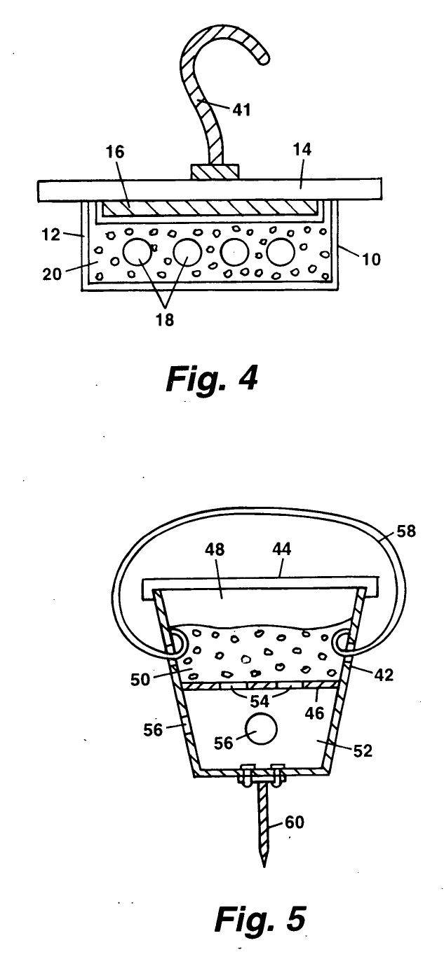

FIG. 4 illustrates an alternative means of positioning dispenser 10 by providing hook41 for locating the invention in a general area where animals are unwanted such as a borough in a yard or near a house foundation.

FIG. 5 is a cross-sectional view of another embodiment of the invention showing a dispenser comprising a pail 42 having a cover 44 and an interior perforated shelf 46. Upper chamber 48 holds an animal repellent chemical 50 and lower chamber 52 further isolates the chemical from the environment. Perforations 54 in shelf 46 and ports 56 in chamber 52 allow allow chemical effluent generated by chemical 50 to escape into the surrounding atmosphere. Handle 58 and/or ground stake 60 are provided to move and secure the dispenser at a desired location.

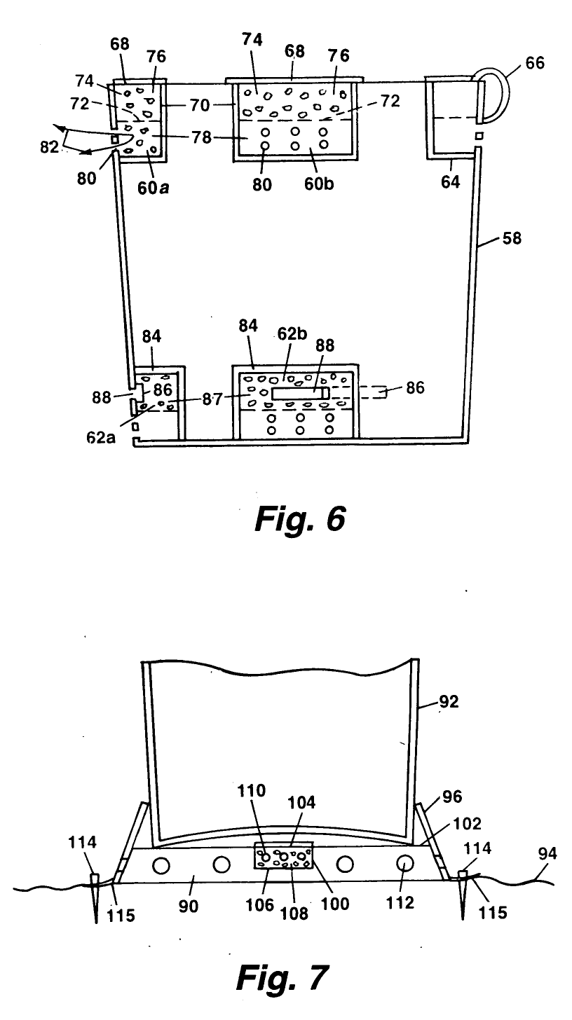

Referring next to FIG. 6, a partially cross-sectional front view of an additional embodiment of the invention is shown in which three variations of a dispenser are disposed in a trashcan or receptacle 58. The cover of can 58 is not shown for purposes of clarity. Two of the dispenser variations, 60 and 62 are molded directly into can 58 and the third dispenser variation 64 is a separate unit and is suspended from the rim of the can by, for example, a wire clip 66.

Dispenser 60 is shown twice as a side view 60a and as a front view 60b and is composed of cover 68 and dispenser body 70 which cover may either snap on and off or slide back and forth to open and close the interior of dispenser body 70. Perforated shelf 72 divides the dispenser into an upper chamber 74 for containing repellent chemical 76 and a lower chamber 78. Ports 80 formed directly in the wall of can 80 allow the repellent effluent, as indicated by arrow 82, to directly enter the atmosphere.

The second variation 62 is also illustrated as two views, as a side view 62a and a front view 62b, and is the same as dispenser 60 except that the cover 84 is not removable. Instead sliding door 86 for the insertion of chemical 87 is arranged to cover port 88 formed directly in the wall of the can.

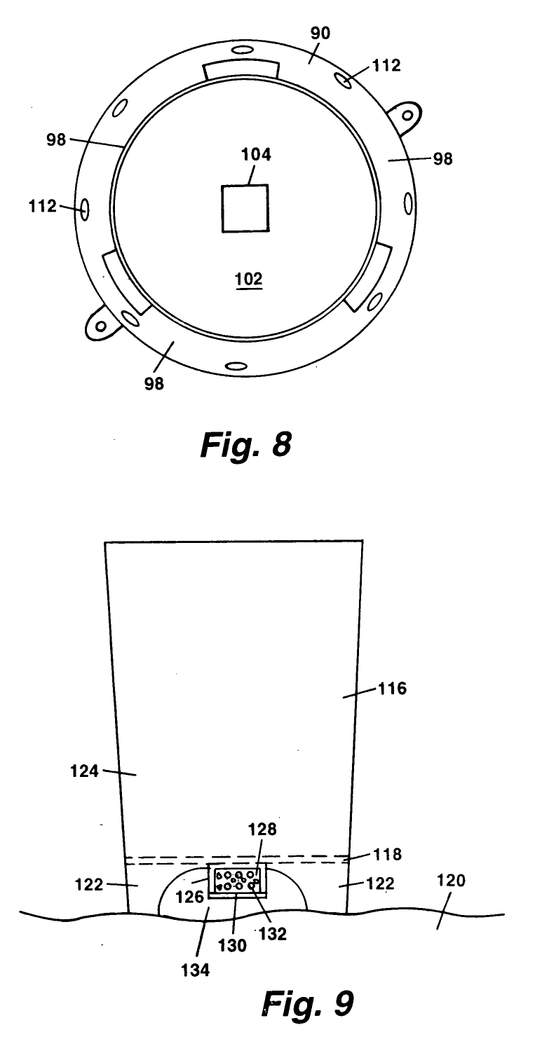

Referring next to FIGS. 7 and 8, a cross-sectional side view and plan view of an additional embodiment of the invention is illustrated. This arrangement comprises a base 90 for supporting a trashcan 92 above ground level 94. Wall 96 extending above base 90 may be added to aid in preventing can 92 from tipping over and may completely surround the can or may be a partial wall disposed in sections 98 about the base to achieve the same purpose.

Dispenser 100 is disposed in top 102 of base 90 and includes removable cover 104 and chamber 106 for containing animal repellent chemical 108. Ports 110 are disposed in chamber 106 and ports 112 are disposed in base 90 to allow the effluent from repellent 108 to disperse to the environment. Base 90 may be held in place by the weight of can 92 alone or may additionally be secured by stakes 114 driven into ground 94 through tabs 114 attached to base 90.

FIG. 9 illustrates a front view of a further embodiment of the invention in which trashcan 116 has a bottom 118 raised above ground 120 by extensions 122 of sidewall 124. Compartment 126 for containing animal repellent chemical 128 is mounted below bottom 118 and has removable cover 130 for inserting chemical 128. Ports 132 allow the chemical effluent to pass into the environment through space 134 at the bottom of the can as created by extensions 122.

As variations in the above-described embodiments will now be obvious to those skilled in the art, the invention is accordingly defmed by the following claims.

What is claimed is:

1. A dispenser for animal repellant effluent comprising in combination:
A. a lower compartment portion to contain said repellant, said compartment having a plurality of side vents to dispense said effluent;
B. an upper cover portion cooperating with said compartment to deposit and protect said repellent; and
C. positioning means attached to said dispenser for positioning said dispenser in a selected location.

2. The dispenser of claim 1 further including a trashcan receptacle and cover and wherein said dispenser is attached to said trashcan cover.

3. The dispenser of claim 2 further including a handle attached to said upper cover portion.

4. The dispenser of claim 1 further including a trashcan receptacle having at least one handle disposed on the side thereof and wherein said dispenser is attached under said handle.

5. The dispenser of claim 1 wherein said positioning means comprises an adhesive strip.

6. The dispenser of claim 1 further including a dumpster and wherein said dispenser is attached to the cover of said dumpster.

7. The dispenser of claim 1 further including a dumpster and wherein said dispenser is attached to the side of said dumpster.

8. The dispenser of claim 1 wherein said positioning means comprises a hook for suspending said dispenser.

9. The dispenser of claim 1 wherein said dispenser comprises a pail and further includes a perforated shelf within said pail for supporting said repellent.

10. The dispenser of claim 9 further including a handle attached to said pail.

11. The dispenser of claim 9 further including a stake attached to said pail.

12. A dispenser for animal repellent effluent comprising in combination:
A. an upper compartment to contain said repellent;
B. a lower compartment communicating with said upper compartment having a plurality of side vents to dispense said effluent;
C. a perforated shelf disposed between said upper and lower compartments; and
D. a removable cover cooperating with said upper compartment for depositing said repellent.

13. The dispenser of claim 12 further including positioning means attached to said dispenser for positioning said dispenser in a selected location.

14. The dispenser of claim 12 further including a trashcan and wherein said upper and lower compartments are integrally molded with and form a structural part of said trashcan.

15. The dispenser of claim 14 further including a sliding door in said trashcan communicating with said upper compartment.

16. A trashcan support and animal repellent effluent dispenser comprising in combination:
A. a substantially circular base member;
B. a rim disposed below said base member for raising said base member above the ground a desired distance, said rim having a plurality of spaced apart rim ports about its circumference;
C. a compartment attached to and mounted below said base member for containing said repellent, said compartment having a plurality of ports for emitting said effluent below said base and through said rim ports to the environment; and
D. a removable cover atop said compartment for depositing said repellent.

17. The dispenser of claim 16 further including a circular wall disposed above and attached to said base for securing said trashcan in an upright position.

18. The dispenser of claim 17 further including at least one tab member extending from the bottom of said rim and further including a stake to engage said tab to secure said dispenser to the ground.

19. A trashcan animal repellent effluent dispenser comprising in combination:
A. a trashcan comprising:
1. a sidewall enclosing the interior of said can; and
2. a bottom member attached to said sidewall;
B. a base formed by the extension of a portion of said sidewall to raise said bottom above ground level and to provide communication with the underside of said bottom member and the environment;
C. a compartment mounted below said bottom for containing said repellent, said compartment having a plurality of ports for emitting said effluent; and
D. a removable cover attached to said compartment for depositing said repellent.
